(19) United States
(12) Patent Application Publication
(10) Pub. No.: US 2026/0020974 A1
(43) Pub. Date: Jan. 22, 2026

(54) MANDIBULAR ADVANCEMENT DEVICE

(71) Applicant: DCSTAR INC, New York, NY (US)

(72) Inventor: David LUO, New York, NY (US)

(21) Appl. No.: 18/776,999

(22) Filed: Jul. 18, 2024

(51) Int. Cl. A61F 5/56

(52) U.S. Cl. CPC A61F 5/566

(57) ABSTRACT

A mandibular advancement device, including an upper tray assembly and a lower tray assembly. The upper tray assembly includes an upper frame and an upper moldable component, while the lower tray assembly includes a lower frame and a lower moldable component. The frames are configured to engage with the moldable components, and the moldable components are configured to at least partially conform to the teeth and/or gums. Furthermore, the frames include at least two materials of different hardness, and the first material is softer compared to the second material. The outer walls of the frames at least partially include the first material, while the bottom walls at least partially include the second material. This design enhances the comfort and safety of the mandibular advancement device, thereby improving the user experience and enhancing therapeutic effectiveness.

### TECHNICAL FIELD

This disclosure pertains to the treatment and improvement of respiratory-related diseases and provides a mandibular advancement device.

### BACKGROUND

Obstructive Sleep Apnea Syndrome (OSAS) is a condition characterized by respiratory pauses during sleep due to obstructive lesions in the upper airway. It is defined as having more than 30 breathing pauses during 7 hours of nighttime sleep, each lasting at least 10 seconds; or an average of more than 5 breathing pauses per hour. OSAS manifests as loud snoring, brief episodes of gasping, and alternating pauses in breathing lasting over 10 seconds during sleep. These pauses can cause the user to choke, sometimes waking up abruptly, and sleep can continue when breathing resumes. Consequently, sleep quality is severely compromised, leading users to often feel fatigued and sleepy during the day, and long-term sufferers may develop symptoms of anxiety and depression. Repeated episodes of hypoxia during sleep can cause long-term damage to various organs and systems, eventually leading to complications in the cardiovascular, respiratory, nervous, endocrine, and urinary systems. OSAS is classified into mild, moderate, and severe degrees based on its severity. The primary goal in treating OSAS is to increase the tension in the respiratory airways to facilitate normal gas exchange, allowing oxygen to be drawn from the external environment and carbon dioxide to be expelled from the body. Typical treatments for OSAS include etiological treatment, adopting healthy lifestyle habits, pharmacotherapy, continuous positive airway pressure (CPAP), mandibular advancement devices (MAD), surgical interventions, and other device-based treatments.

Among these methods, CPAP therapy is often the first choice for many individuals. It involves the use of a CPAP machine that provides a constant and stable air pressure via a hose and mask, maintaining the airways in a positive pressure state throughout the breathing cycle to prevent collapse and keep the airways open. However, some individuals cannot tolerate or adapt to the positive airflow provided by CPAP machines. Moreover, CPAP requires components like hoses and masks that are attached to the user's nose, face, or mouth, making it difficult to fall asleep and affecting sleep quality. Additionally, CPAP machines are relatively bulky and not easily portable, making it more difficult for users to travel with them. The use of numerous components in CPAP therapy also leads to complex and laborious cleaning routines. Improper cleaning may result in bacterial or fungal infections, causing other respiratory diseases.

Surgical treatment of OSAS has also been popular. When the cause of obstruction is clear, surgeries such as removal of nasal polyps, correction of deviated nasal septum, tonsillectomy, and adenoidectomy can alleviate the obstruction; orthognathic surgery for oral correction is one treatment method for OSAS caused by jaw factors. Due to the advancement of the maxilla and mandible, parameters such as the volume and minimum cross-sectional area of the user's upper airway post-surgery are significantly increased compared to pre-surgery, markedly improving OSAS symptoms, and in some cases, completely eliminating them. However, surgical treatments carry risks, including postoperative bleeding, infection, and poor surgical outcomes.

Therefore, treatment with CPAP and/or surgery may not be the best approach for OSAS.

Treatment with a MAD works by indirectly enlarging the upper airway through forward movement of the mandible, achieving therapeutic effects. MAD treatments can be categorized into three types based on the area of effect: soft palate lifters (SPL), tongue retaining devices (TRD), and mandibular advancement devices. SPL fix the device to the upper teeth and extend backward, directly affecting the soft palate and uvula, reducing their drooping, reducing contact between an elongated soft palate and uvula with the base of the tongue and posterior pharyngeal wall, thus preventing pharyngeal narrowing or blockage; they also reduce vibrations of the soft palate, thereby lessening snoring. However, because touching the soft palate can easily induce nausea and requires long-term training to tolerate, it is seldom used. TRD work by creating a vacuum in the bulb at the front of the device to pull the tongue forward, preventing the tongue from falling back and thus widening the airspace behind the tongue and soft palate. However, TRD can cause discomfort and are poorly secured, and users have poor tolerance to them, all of which lead to rare use of this method. The mandibular advancement device functions by maintaining the mandible in a protruded position, enlarging the oropharyngeal cavity, and indirectly moving the tongue forward, which widens the airway behind the tongue. It also reduces the backward pressure of the tongue on the soft palate, thereby broadening the airway behind the soft palate. Commonly used are adjustable mandibular advancement devices; if the therapeutic effect is inadequate, the distance of mandibular advancement can be increased to widen the airway; if discomfort occurs during treatment, the extent of advancement can be reduced. These adjustable devices not only enhance user tolerance but also achieve effective therapeutic outcomes.

However, designing mandibular advancement devices poses multiple challenges: (1) Stability: During sleep, the user's movements or unconscious jaw clenching may cause the device to dislodge, thus affecting the sustainability of the treatment; (2) Applicability: Due to the varying shapes and sizes of individuals' mouths and teeth, and the high costs associated with customizing devices, designing a device that is suitable for the majority presents significant challenges; (3) Comfort: When biting on the mandibular advancement device, the inside of the cheeks and tongue can easily scrape against the device, leading to injuries. Prolonged wearing of the device and extended bite time can also cause dental discomfort.

To overcome these challenges and introduce a more stable, applicable, and comfortable mandibular advancement device, a comprehensive design approach is necessary. This includes, but is not limited to, considering material selection to ensure the device provides sufficient strength and comfort, while also having the appropriate flexibility to adapt to different oral structures. By taking these factors into account comprehensively, the therapeutic effectiveness and comfort of the mandibular advancement device can be enhanced, better meeting the needs of users.

### SUMMARY

To resolve the aforementioned technical issues, a mandibular advancement device is provided. The mandibular advancement device includes an upper tray assembly and a lower tray assembly. The mandibular advancement device is configured to prevent or reduce snoring and/or obstructive sleep apnea during sleep. The upper tray assembly is configured to be adjacent to a portion of an upper dental arch of a user and includes an upper frame and an upper moldable component. The lower tray assembly is configured to be adjacent to a portion of a lower dental arch of the user and includes a lower frame and a lower moldable component. The upper frame and the lower frame are each configured to engage and support the upper moldable component and lower moldable component respectively. The upper moldable component and the lower moldable component are configured to at least partially conform to teeth and/or gums. The upper frame and the lower frame include at least two materials of different hardness, with the hardness of a first material being less than the hardness of a second material. The upper frame and the lower frame each have at least one bottom wall. The at least one bottom wall of the upper frame has a first end and a second end, between which at least part of the at least one bottom wall forms an approximate arc. The at least one bottom wall of the lower frame has a third end and a fourth end, between which at least part of the at least one bottom wall forms an approximate arc.

In one embodiment, the shape of the upper tray assembly is approximately arcuate, U-shaped, or arch-shaped.

In one embodiment, the shape of the lower tray assembly is approximately arcuate, U-shaped, or arch-shaped.

In one embodiment, the first end, the second end, the third end, and the fourth end include the first material.

In one embodiment, the hardness of the second material is greater than the hardness of the first material.

In another embodiment, a mandibular advancement device is provided. The mandibular advancement device includes an upper tray assembly and a lower tray assembly. The mandibular advancement device is configured to prevent or reduce snoring and/or obstructive sleep apnea during sleep. The upper tray assembly is configured to be adjacent to a portion of an upper dental arch of a user and includes an upper frame and an upper moldable component. The lower tray assembly is configured to be adjacent to a portion of a lower dental arch of the user and includes a lower frame and a lower moldable component. The upper frame and the lower frame are each configured to engage and support the upper moldable component and lower moldable component respectively. The upper frame and the lower frame include at least two materials of different hardness, with the hardness of a first material being less than the hardness of a second material. The upper frame and the lower frame each have at least one outer wall, and the at least one outer wall of each frame at least partially includes the first material.

In one embodiment, the at least one outer wall includes only the first material.

In one embodiment, in a portion of the at least one outer wall configured to be in contact with an oral cavity, an area of the first material is greater than an area of the second material.

In one embodiment, the first material includes silicone rubber, polyvinyl alcohol, polyurethane, ethylene-vinyl acetate, thermoplastic olefin, thermoplastic elastomer, thermoplastic rubber, polylactic acid, and polylactic-co-glycolic acid.

In one embodiment, the second material includes polycarbonate, polypropylene, acrylonitrile butadiene styrene, polyethylene, polymethyl methacrylate, polyurethane, stainless steel, titanium alloy, nickel-titanium alloy, and aluminum alloy.

In one embodiment, the at least one outer wall forms an angle of between 20° to 150° with a horizontal plane.

In yet another embodiment, a mandibular advancement device is provided. The mandibular advancement device includes an upper tray assembly and a lower tray assembly. The mandibular advancement device is configured to prevent or reduce snoring and/or obstructive sleep apnea during sleep. The upper tray assembly is configured to be adjacent to a portion of an upper dental arch of a user and includes an upper frame and an upper moldable component. The lower tray assembly is configured to be adjacent to a portion of a lower dental arch of the user and includes a lower frame and a lower moldable component. The upper frame and the lower frame are each configured to engage and support the upper moldable component and lower moldable component respectively. The upper frame and the lower frame include at least two materials of different hardness, with the hardness of a first material being less than the hardness of a second material. The upper frame and the lower frame each have at least one bottom wall, and the at least one bottom wall of each frame at least partially includes the second material.

In one embodiment, the at least one bottom wall includes only the second material.

In one embodiment, the thickness of the second material of the at least one bottom wall is at least 0.3 mm.

In one embodiment, the projected area of the at least one bottom wall on a horizontal plane is at least 9 cm².

In one embodiment, the first material and the second material are joined by methods including crimping, over-molding, snap-fitting, bonding, thermal fusion, screws, and hooks.

In another embodiment, a mandibular advancement device is provided. The mandibular advancement device includes an upper tray assembly and a lower tray assembly. The mandibular advancement device is configured to prevent or reduce snoring and/or obstructive sleep apnea during sleep. The upper tray assembly is configured to be adjacent to a portion of an upper dental arch of a user and includes an upper frame and an upper moldable component. The lower tray assembly is configured to be adjacent to a portion of a lower dental arch of the user and includes a lower frame and a lower moldable component. The upper frame and the lower frame are each configured to engage and support the upper moldable component and lower moldable component respectively. The upper frame and the lower frame include at least two materials of different hardness, with the hardness of a first material being less than the hardness of a second material. The upper frame and the lower frame each have at least one bottom wall and at least one outer wall, and the upper frame and the lower frame have one or more of the following characteristics: a). a volume ratio of the first material to the second material between 0.05 to 20; b). a thickness of at least part of the at least one outer wall between 0.3 mm to 6 mm; and c). a thickness of at least part of the at least one bottom wall between 0.3 mm to 6 mm.

In one embodiment, a height of the middle of the at least one outer wall is greater than the height at the ends of the at least one outer wall.

In one embodiment, the width of the middle of the at least one outer wall is less than the width at the ends of the at least one outer wall.

In one embodiment, the upper frame includes a first outer wall and a second outer wall, and the lower frame includes a third outer wall and a fourth outer wall.

In one embodiment, the second outer wall includes only the first material, and the first outer wall includes the second material.

In one embodiment, the third outer wall includes only the first material, and the fourth outer wall includes the second material.

The implementation of a mandibular advancement device provided by this disclosure at least includes the following benefits:
1. Existing mandibular advancement devices on the market typically use a frame made from a single material. Frames made solely from rigid materials can scrape against the inside of the mouth during wear, causing irritation or even injury to the soft tissues or the tongue. Additionally, if the entire mandibular advancement device is made from moldable material or other softer materials, it may not provide sufficient support during biting. Moreover, the lower tray assembly needs to move forward relative to the upper tray assembly, and they must be fixed together. Softer materials may deform, potentially changing the forward distance, which can lead to separation of the upper and lower tray assemblies and affect the treatment outcome. To address this issue, this disclosure adopts an innovative design: the frame includes at least two materials of different hardness. The outer wall of the frame is primarily made from the first material, which is softer and helps reduce scraping against the inside of the mouth during wear. The bottom wall of the frame is at least partially made from the second material, which is harder and provides the necessary support and stability during the biting process. Furthermore, the outer wall of the frame forms a certain angle with the horizontal plane to accommodate the natural inclination of the front teeth towards the lip side, fitting the shape of the teeth more closely. This design not only ensures support during the biting process but also securely connects the upper and lower tray assemblies, reducing the possibility of the device falling out, thereby ensuring good therapeutic effects during treatment.
2. When the user wears the mandibular advancement device, the lower tray assembly moves forward relative to the upper tray assembly to achieve the effect of mandibular advancement. However, this advancement can lead to crowding inside the mouth, especially when the forward distance is large, potentially causing contact with the deeper parts of the mouth and the frame, leading to a sensation of soreness and foreign body presence. Therefore, the outer walls of the frame are mostly made from the first material. Compared to traditional rigid materials, this first material is softer and more deformable, allowing it to better conform to the internal shape of the mouth, thus providing a more comfortable wearing experience. Additionally, the ends of the frame also use the softer first material to ensure comfort around the jaw joints and/or deeper parts of the mouth. This design helps enhance the comfort of wearing the mandibular advancement device, reduces discomfort for the user, improves the user experience, and thereby better enhances the therapeutic effects.
3. Mandibular advancement devices typically need to be worn for extended periods during sleep, and prolonged wearing and biting can cause discomfort or soreness in the user's mouth or teeth. Additionally, during the use of the mandibular advancement device, the lower tray assembly moves forward relative to the upper tray assembly to achieve the mandibular advancement effect. During this forward movement, the lower dental arch is subjected to forward traction forces, while the lower tray assembly experiences a reactive force from the lower dental arch's tendency to return to its original position; similarly, the upper dental arch is subjected to backward traction forces, while the upper tray assembly experiences a reactive force from the upper dental arch's tendency to return to its original position. Consequently, the larger force-bearing surface of the lower tray assembly is oriented towards the tongue side, and the larger force-bearing surface of the upper tray assembly is oriented towards the lip side. The present disclosure offers an improved frame design, and the outer wall of the frame is made from different materials depending on the direction of the force, with most parts that are likely to contact soft tissues of the mouth being made from the softer first material. Compared to the traditional design that uses a single rigid material, this design provides a more comfortable force-bearing surface, alleviating discomfort in the mouth and teeth, thereby enhancing the user's experience.
4. Existing mandibular advancement devices on the market still suffer from issues of dislodgement, with numerous user feedback indicating that the devices frequently separate from the mouth during sleep, leading to the device falling off. Testing and analysis have revealed that this dislodgement issue is primarily due to the requirement for the lower tray assembly to move forward relative to the upper tray assembly, and the possibility of the user moving or unconsciously opening their mouth during sleep, which can cause the mandibular advancement device to dislodge. Moreover, the outer walls of market-available frames are mostly made from rigid materials, which tend to slide easily upon contact with the mouth, further increasing the likelihood of dislodgement. To address this issue, the present disclosure provides an improved frame design with an outer wall that includes the softer first material. The first material has a higher coefficient of friction compared to traditional rigid materials, thus increasing friction with the mouth. By increasing the friction, the mandibular advancement device can be more securely fixed in the mouth, reducing the likelihood of dislodgement, and enhancing the stability and sustainability of the device during sleep.

### BRIEF DESCRIPTION OF THE DRAWINGS

FIG. 1 is a schematic structural diagram of a mandibular advancement device in accordance with one embodiment;

FIG. 2 is an exploded structural diagram of a mandibular advancement device in accordance with multiple embodiments;

FIG. 3 is a schematic diagram of the horizontal, sagittal, and coronal planes in accordance with multiple embodiments;

FIG. 4 is a top view of the upper frame and lower frame in a separated form in accordance with multiple embodiments;

FIG. 5 is a cross-sectional view of the upper frame along the A-A direction in FIG. 4 in accordance with one embodiment;

FIG. 6 is a rear view of the upper frame and lower frame in a combined form in multiple embodiments;

FIG. 7 is a cross-sectional view of the frame along the D-D direction in FIG. 6 in accordance with one embodiment;

FIG. 8 is a cross-sectional view of the upper frame along the B-B direction in FIG. 4 in accordance with one embodiment;

FIG. 9 is a partial sectional schematic diagram of the angle between the outer wall of the frame and the horizontal plane in accordance with one embodiment;

FIG. 10 is a sectional schematic diagram of the upper frame along the A-A direction and the C-C direction in FIG. 4 in accordance with one embodiment;

FIG. 11 is a partial cross-sectional schematic diagram of the outer wall of the frame in accordance with one embodiment;

FIG. 12 is a partial cross-sectional schematic diagram of the outer wall of the frame in accordance with one embodiment;

FIG. 13 is a partial cross-sectional schematic diagram of the outer wall of the frame in accordance with one embodiment.

### DETAILED DESCRIPTION

To elucidate the objectives, features, and advantages of this disclosure more clearly, a detailed description of specific embodiments of the disclosure is provided below in conjunction with the accompanying drawings. Many specific details are provided in this description to facilitate a comprehensive understanding of the disclosure. However, it is feasible to implement the disclosure in various ways other than those described here, and one skilled in the art can make similar modifications without departing from the essence of the disclosure. Therefore, the disclosure is not confined to the specific embodiments disclosed herein.

This disclosure pertains to a mandibular advancement device that addresses the limitations of most frames of mandibular advancement devices on the market, which include a single material. To resolve this issue, the disclosure adopts an innovative design—the frame of the mandibular advancement device includes two materials of different hardness. This design enhances the comfort and safety of the mandibular advancement device, thereby improving the user experience and enhancing the therapeutic effects.

The disclosure provides a mandibular advancement device 1 configured to prevent or reduce snoring and/or obstructive sleep apnea during sleep, including an upper tray assembly 2 and a lower tray assembly 3. The upper tray assembly 2 is configured to be adjacent to a portion of the user's upper dental arch, including an upper frame 21 and an upper moldable component 22, primarily functioning to support the teeth of the upper jaw. The lower tray assembly 3 is configured to be adjacent to a portion of the user's lower dental arch, including a lower frame 31 and a lower moldable component 32, primarily functioning to support the teeth of the lower jaw. The dental arches generally have an arch shape that conforms to the natural arrangement of the user's teeth. Specifically, both the upper dental arch and the lower dental arch form a roughly symmetrical and approximately arch-shaped curvature. Therefore, the upper tray assembly 2 and the lower tray assembly 3 are shaped approximately in arc, U-shape, or arch, conforming to the general arch-shaped curve of the user's dental arches. Together, the upper tray assembly 2 and the lower tray assembly 3 form the mandibular advancement device 1, which supports the upper and lower dental arches, adjusts their anterior and posterior distances, and applies mild pressure to the teeth to achieve the therapeutic effect for OSAS. In this disclosure, the frames 21 and 31 include an upper frame 21 and a lower frame 31, while the moldable components 22 and 32 include an upper moldable component 22 and a lower moldable component 32. The mandibular advancement device 1, when positioned in the user's oral cavity, is configured to have a side nearer the lip (the lip side) and a side nearer the tongue (the tongue side) in the front-back direction.

The upper moldable component 22 and the lower moldable component 32 are configured to at least partially conform to the teeth and/or gums. The moldable components 22 and 32 are typically arc-shaped, U-shaped, or arch-shaped, similar to the shape of the dental arch, ensuring that moldable components 22 and 32 fully contact the teeth and gums. Parts of the moldable components 22 and 32 may include raised or recessed designs to accommodate different tooth sizes and shapes. Typically, the moldable components 22 and 32 include elastic materials such as silicone rubber, polyurethane, polyvinyl alcohol, ethylene-vinyl acetate, etc., which must be biocompatible, soft, and comfortable, causing no damage to the oral cavity and suitable for long-term wear inside the mouth.

There are several different types of moldable components available on the market. Commonly, these are custom-made in hospitals based on impressions of the user's teeth, allowing for precise customization of the moldable components to better fit the user's teeth, thereby ensuring greater comfort and optimal therapeutic effects. However, custom moldable components require specialized technology and equipment, involve higher costs, and have longer production times. Additionally, if significant adjustments or changes are needed after the moldable components are made, they can be challenging and may require remaking or reprocessing. Therefore, thermoplastic moldable components are also popular in the market. The manufacturing process for thermoplastic moldable components is relatively simple: the material only needs to be heated to soften, then molded by biting to create the desired shape, and it can be reheated for minor adjustments after setting. This method is faster and reduces costs. However, thermoplastic moldable components have lower precision and poorer adaptability due to the simplicity of the production process and the lack of professional equipment. Additionally, there are limited choices for materials, as it requires special thermoplastic materials, but these materials might have limitations in terms of hardness and comfort. The moldable components 22 and 32 provided in this disclosure include, but are not limited to, customized moldable components and thermoplastic moldable components.

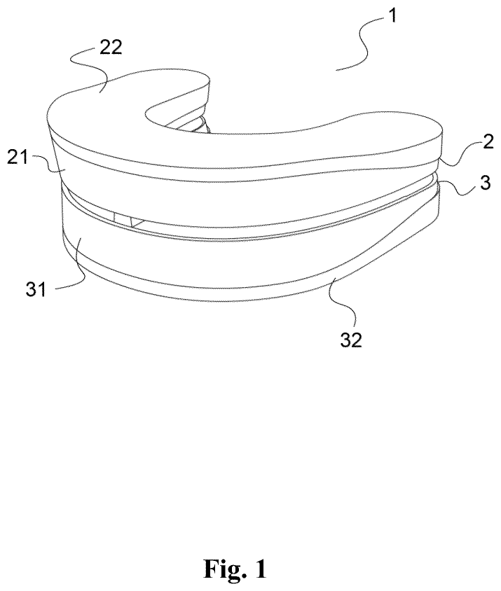

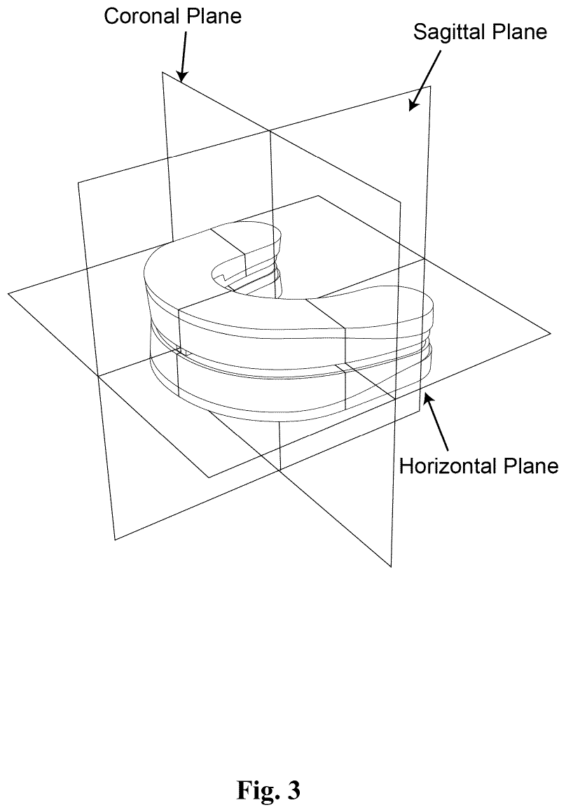

Specifically, referring to FIGS. 1 to 4, the upper frame 21 and the lower frame 31 are each configured to engage and support the upper moldable component 22 and the lower moldable component 32 respectively, providing stable support within the user's mouth. They may also have adjustment features that allow for modifications based on the user's needs. The shapes of frames 21 and 31 typically resemble those of moldable components 22 and 32 and are similarly shaped in approximate arcs, U-shapes, or arches to ensure a fit that conforms to the internal shape of the teeth in the mouth.

The upper frame 21 and the lower frame 31 include at least two materials of different hardness, and the hardness of the first material 4 is lower than that of the second material 5. The first material 4 is usually a soft and elastic material, including but not limited to silicone rubber, polyvinyl alcohol, polyurethane, ethylene-vinyl acetate, thermoplastic olefin, thermoplastic elastomer, thermoplastic rubber, polylactic acid, and polylactic-co-glycolic acid. The first material 4 typically has good biocompatibility, can contact the inside of the mouth without causing allergies or other adverse reactions. As the first material 4 is softer compared to traditional rigid materials, it is more comfortable and suitable for prolonged wear and biting, helping to reduce discomfort and oral irritation for the user. Moreover, although the first material 4 is relatively soft, it also has a certain degree of abrasion resistance, capable of withstanding the tension and friction in the mouth during wear, thereby improving the overall component's lifespan. The hardness of the second material 5 is higher than that of the first material 4 and usually has stronger hardness, able to withstand greater pressure, ensuring that frames 21 and 31 are not easily deformed or damaged during wear and biting, maintaining structural stability. The second material 5 includes, but is not limited to, polycarbonate, polypropylene, acrylonitrile butadiene styrene, polyethylene, polymethyl methacrylate, polyurethane, stainless steel, titanium alloy, nickel-titanium alloy, and aluminum alloy.

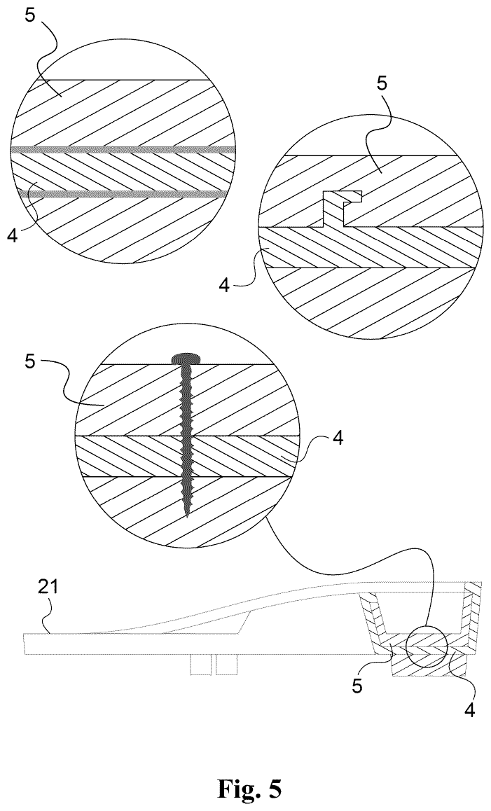

As shown in FIG. 5, the first material 4 and the second material 5 on the frames 21 and 31 are typically fixed in a non-detachable manner. Various methods can be employed for this fixation, including crimping (which involves applying pressure to tightly combine the two materials), over-molding, snap-fitting (where snaps are constructed on both the first material 4 and the second material 5 for joining), bonding (e.g., adhesive bonding), thermal fusion (where the first material 4 is heated until it melts and then fixed upon cooling on the second material 5), or using additional fixing accessories such as screws and hooks. The connection method may use one form, or even combine two or more forms, to achieve the function of connecting and/or fixing the first material 4 and the second material 5. The choice of the appropriate connection method depends on specific design requirements, material characteristics, and the usage environment to ensure the connection is firm, reliable, durable, and easy to assemble during production, as well as convenient for users to adjust. The preferred fixing method is over-molding. Over-molding can tightly secure the first material 4 and the second material 5 together, providing a more stable fixing effect, preventing the materials from falling apart or loosening. Additionally, it prevents external substances from entering or remaining in the gaps between materials, effectively protecting the interior from residual bacteria or cleaning residues and prolonging the lifespan.

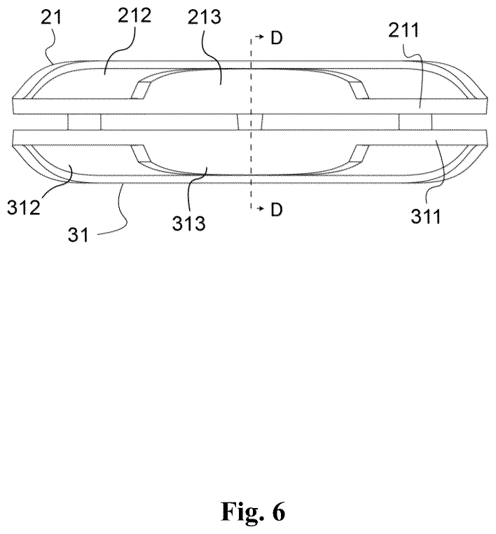

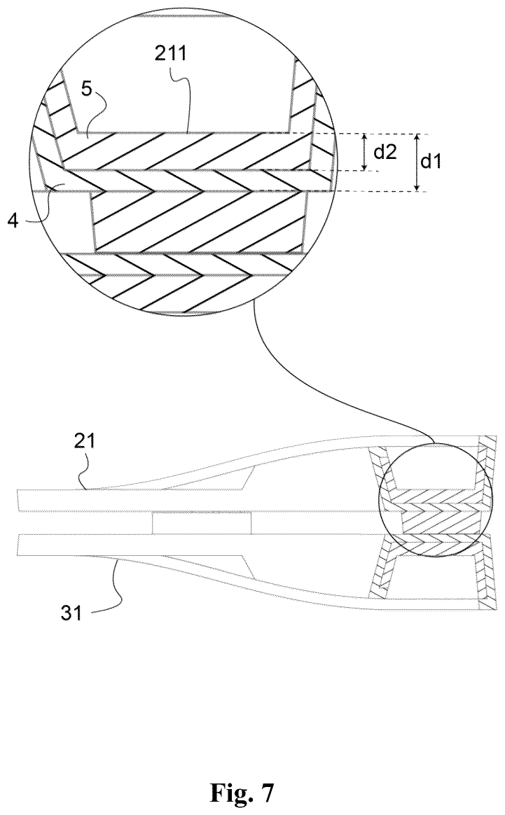

The upper frame 21 and the lower frame 31 include bottom walls 211 and 311. As shown in FIGS. 6 and 7, the bottom walls 211 and 311 are generally horizontal, with the tops of the bottom walls 211 and 311 in contact with the moldable components 22 and 32. The bottom of one bottom wall faces the opposite bottom of another bottom wall and the two together form at least one channel. When users wear the mandibular advancement device 1, the device does not completely close, allowing external airflow to enter the user's mouth through the channel, ensuring the free flow of air inside the mouth. This also meets the needs of users who habitually breathe through their mouths during sleep, avoiding the risk of suffocation. If thermoplastic moldable components 22 and 32 are used, which require shaping by biting the upper tray assembly 2 and the lower tray assembly 3, the lack of sufficient vertical support might make the shaping process difficult or even unsuccessful. Furthermore, since the lower tray assembly 3 needs to move forward relative to the upper tray assembly 2, and the upper and lower frames 21 and 31 need to be fixed to each other. If softer materials are used, they can deform easily, causing changes in the forward distance, which not only affects the therapeutic effect but also causes the upper and lower frames 21 and 31 to separate easily. Therefore, this disclosure provides an improved design for frames 21 and 31, with the bottom walls 211 and 311 at least partially including the second material 5, providing sufficient vertical support for the moldable components 22 and 32. The projected area of the bottom walls 211 and 311 on the horizontal plane is at least 9 cm², with a thickness of at least part of the bottom walls between 0.3 mm to 6 mm, as shown by d1 in FIG. 7. The thickness of the second material 5 included in the bottom walls 211 and 311 is at least 0.3 mm, as shown by d2 in FIG. 7. The overall weight of frames 21 and 31 is between 3 g to 50 g, with the volume ratio of the first material 4 to the second material 5 ranging from 0.05 to 20. Preferably, the bottom walls 211 and 311 include only the second material 5, and the second material has a thickness of between 1 mm to 2 mm. This design ensures both the necessary support and rigidity required by the mandibular advancement device 1 and avoids making the frames 21 and 31 too thick and heavy, thereby reducing the weight of the mandibular advancement device 1 and lessening the burden on the mouth.

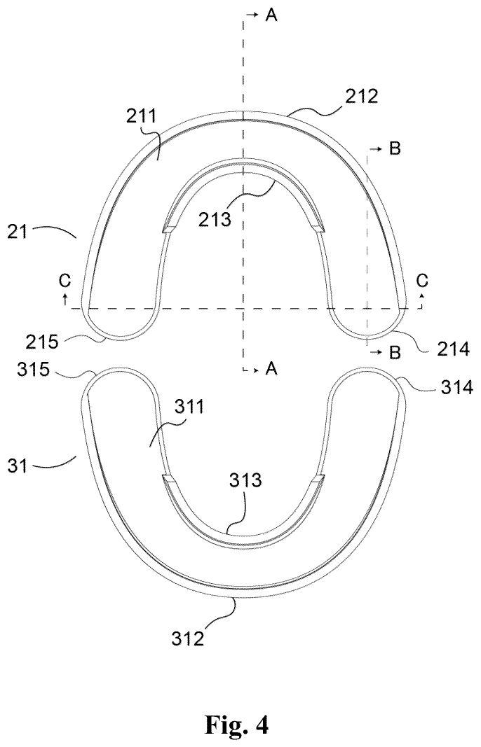

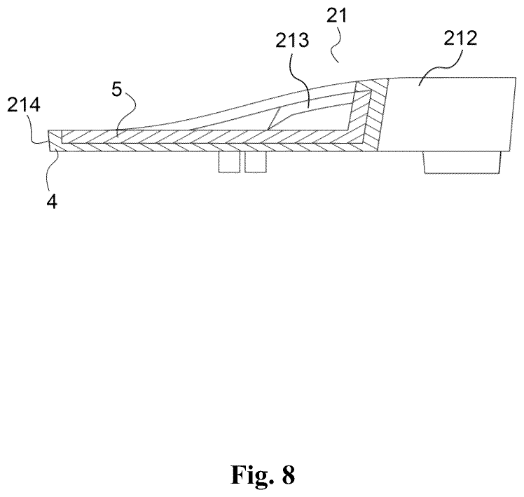

As shown in FIGS. 4 and 8, the bottom wall 211 of the upper frame has a first end 214 and a second end 215, and between these ends, at least part of the bottom wall forms an approximate arc, U-shape, or arch shape; similarly, the bottom wall 311 of the lower frame has a third end 314 and a fourth end 315, with at least part between these ends also forming an approximate arc, U-shape, or arch shape. When users operate the mandibular advancement device 1, the lower tray assembly 3 moves forward relative to the upper tray assembly 2 to achieve the effect of advancing the mandible. However, this forward movement may cause crowding inside the mouth, especially when the forward distance is large, which can result in contact between the deeper parts of the mouth and the frames 21 and 31, causing sensations of soreness and the presence of a foreign object. Therefore, the ends 214, 215, 314, and 315 of the frames are made from the softer first material 4 to ensure comfort in the jaw joints and/or the deeper parts of the mouth. This design helps to enhance the comfort of using the mandibular advancement device 1 and alleviate discomfort for the user, thereby improving the user experience and enhancing the therapeutic effects.

The upper frame 21 and the lower frame 31 include outer walls 212, 213, 312, and 313. As shown in FIGS. 6 to 8, the outer walls 212, 213, 312, and 313 at least partially include the relatively soft first material 4. In the areas of these outer walls that contact the mouth, the area of the first material 4 is greater than that of the second material 5. Compared to traditional rigid materials, this first material 4 is more deformable and can better conform to the internal shape of the mouth, thus providing a more comfortable wearing experience.

During the use of the mandibular advancement device 1, the lower tray assembly 3 advances relative to the upper tray assembly 2 to facilitate the forward movement of the mandible. During this process, the lower dental arch is subjected to a forward traction force, while the lower tray assembly 3 experiences a reactive force from the lower dental arch's attempt to return to its original position. Conversely, the upper dental arch receives a backward traction force, and the upper tray assembly 2 experiences a reactive force from the upper dental arch's attempt to return to its original position. Consequently, the force-bearing surface of the lower tray assembly 3 is oriented towards the tongue side, while the force-bearing surface of the upper tray assembly 2 is positioned towards the lip side. Additionally, since the displacement of the upper tray assembly 2 relative to the original position of the teeth is smaller, the reactive force on the upper tray assembly 2 is less than that on the lower tray assembly 3. Existing mandibular advancement devices on the market often have issues with dislodgement. Many users report that during sleep, these devices frequently separate from the mouth, leading to the device falling out. Testing and analysis have shown that this dislodgement issue is primarily due to the necessary forward movement of the lower tray assembly relative to the upper tray assembly, coupled with potential user movements or unconscious mouth opening during sleep. The lack of sufficient support from the frames' outer walls can also contribute to this problem. Moreover, most frames on the market are made of rigid materials, which, upon contact with the oral cavity, are prone to slipping, further increasing the likelihood of dislodgement.

To address these issues, the frames 21 and 31 in this disclosure include outer walls 212, 213, 312, and 313, providing ample support for the mandibular advancement device 1. Moreover, the parts of these outer walls that contact the oral cavity are primarily made from the first material 4. This first material 4 has a higher coefficient of friction than traditional rigid materials, thereby increasing the friction with the oral cavity. By enhancing the friction, the mandibular advancement device 1 can be more securely fixed within the mouth, reducing the likelihood of dislodgement and improving the device's stability and sustainability during sleep. This design enhances the comfort of using the mandibular advancement device 1, alleviates discomfort for the user, and improves the user experience, thereby better enhancing the therapeutic effects.

Detailed embodiments are presented below to elucidate the configurations of mandibular advancement device 1 provided in this disclosure.

### Embodiment 1

In this embodiment, the mandibular advancement device 1 includes an upper tray assembly 2 and a lower tray assembly 3. The upper tray assembly 2, configured to be adjacent to a portion of the user's upper dental arch, includes an upper frame 21 and an upper moldable component 22. The lower tray assembly 3, configured to be adjacent to a portion of the user's lower dental arch, includes a lower frame 31 and a lower moldable component 32. In this embodiment, the frames 21 and 31 include an upper frame 21 and a lower frame 31, and the moldable components 22 and 32 include an upper moldable component 22 and a lower moldable component 32.

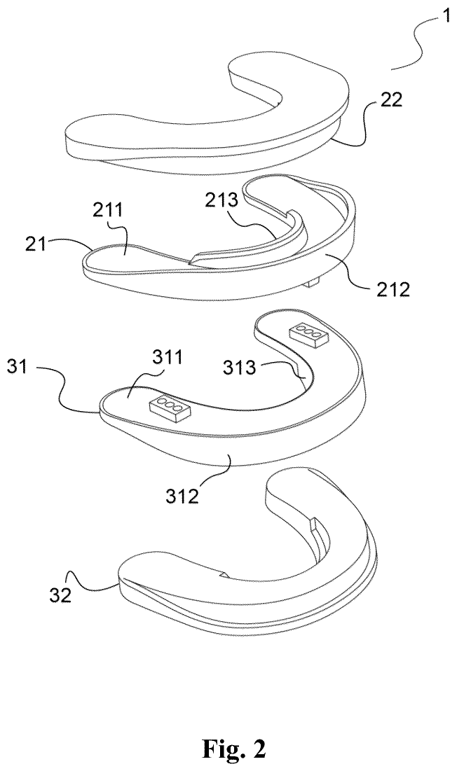

Specifically, as shown in FIG. 2, the upper moldable component 22 and the lower moldable component 32 are configured to at least partially conform to the teeth and/or gums. The upper frame 21 and the lower frame 31 are each configured to engage and support the upper moldable component 22 and the lower moldable component 32, respectively. The upper frame 21 and the lower frame 31 include at least two materials of different hardness, and the hardness of the first material 4 is lower than that of the second material 5.

The upper frame 21 and the lower frame 31 include bottom walls 211, 311, and outer walls 212, 213, 312, 313. As shown in FIGS. 4 and 6, the upper frame 21 includes a bottom wall 211, a first outer wall 212, and a second outer wall 213, while the lower frame 31 includes a bottom wall 311, a third outer wall 312, and a fourth outer wall 313. The first outer wall 212 and the third outer wall 312 are configured to be oriented towards the lip side, and the second outer wall 213 and the fourth outer wall 313 are configured to be oriented towards the tongue side.

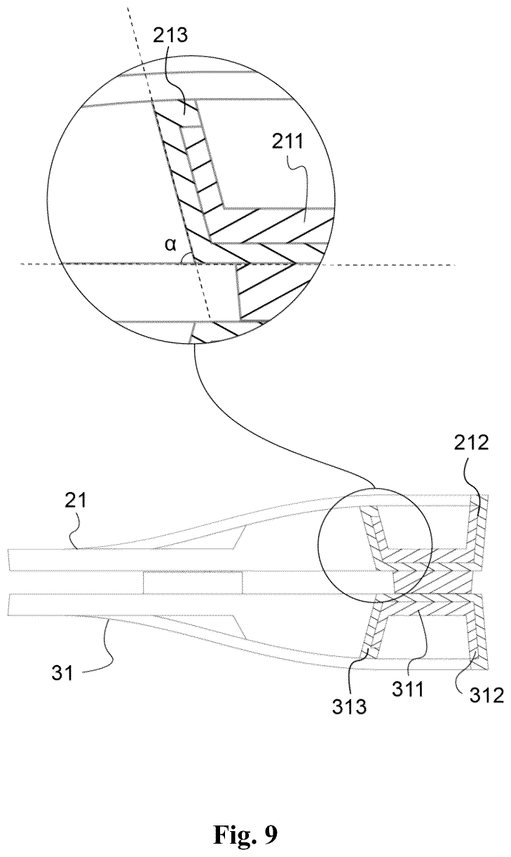

In this embodiment, the outer walls 212, 213, 312, 313 are connected to the bottom walls 211, 311, respectively, as shown in FIG. 9. The upper frame 21 accommodates and secures the moldable component 22 through a structured arrangement of the first outer wall 212, the bottom wall 211, and the second outer wall 213. This arrangement is deliberate: the first outer wall 212 connects to the bottom wall 211, which in turn connects to the second outer wall 213. The first and second outer walls are positioned opposite each other, collectively forming a recessed shape to match the moldable component 22. Similarly, the lower frame 31 accommodates and secures the moldable component 32 through the third outer wall 312, the bottom wall 311, and the fourth outer wall 313. Each tooth has a certain angle difference and is not necessarily perpendicular to the horizontal plane. For example, the anterior teeth (central and lateral incisors) are usually inclined towards the lip at an angle of about 10° to 25° from the vertical plane; the canine teeth (towards the lip side) typically have an inclination angle between 5° to 15°; and the angle of inclination for molars (towards the lip side) is usually between 0 to 5°. The incisors and canines have larger angles relative to the vertical plane, while molars have smaller angles, resulting in different support forces required for different teeth during the biting process. Therefore, the outer walls 212, 213, 312, 313 form an angle of between 20° to 150° with the horizontal plane (oriented away from the bottom walls 211, 311), denoted as angle α in FIG. 9, where preferably, the angle of the outer walls 212, 213, 312, 313 with the horizontal plane is between 45° to 90°

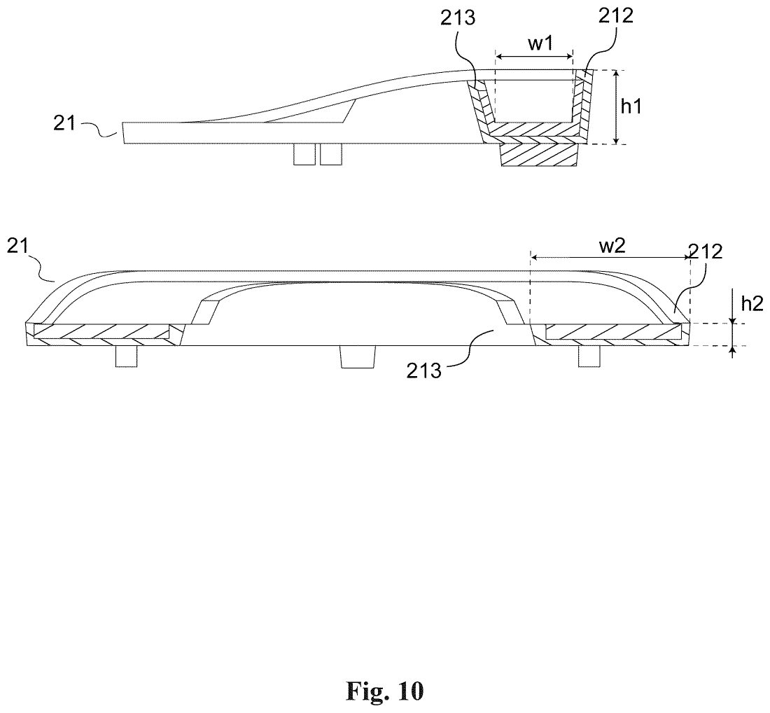

Considering the different positions and sizes of teeth within the oral cavity, the heights and widths of the outer walls 212, 213, 312, 313 are adjusted accordingly. The central incisor region is typically narrower and longer, suited for cutting and tearing food, while the molar region is usually wider and thicker, better suited for chewing and grinding food. Thus, as shown in FIG. 10, the outer walls 212, 213, 312, 313 are typically higher and have a narrower front-to-back distance (which refers to the distance between the first outer wall 212 and the second outer wall 213 or between the third outer wall 312 and the fourth outer wall 313) in the middle (where they intersect with the mid-sagittal plane). The ends of the outer walls 212, 213, 312, 313 are usually lower and have a wider front-to-back distance. This means that the height of the middle (h1 in FIG. 10) of the outer walls 212, 213, 312, 313 is greater than the height at the ends (h2 in FIG. 10), and the width (w1 in FIG. 10) in the middle is less than the width at the ends (w2 in FIG. 10). The higher, narrower middle portion can accommodate and secure the narrower, longer central incisors. The molar areas, although also long, are wider and thicker, having the ends lower and wider avoids the overly wide molars to press against the outer walls 212, 213, 312, 313.

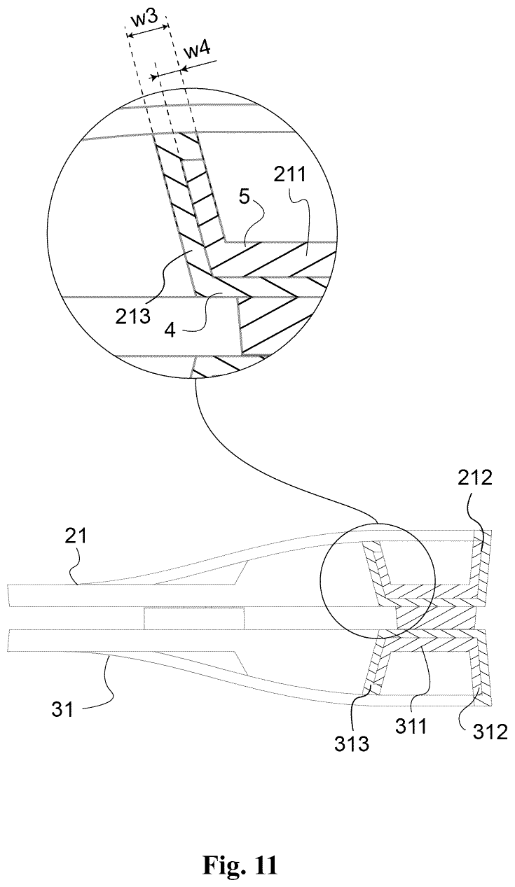

During the use of the mandibular advancement device 1, the lower tray assembly 3 advances relative to the upper tray assembly 2 to facilitate the forward movement of the mandible, as shown in FIG. 11. During this process, the lower dental arch is subjected to a forward traction force, while the lower frame 31 experiences a reactive force from the lower dental arch's attempt to return to its original position. Conversely, the upper dental arch receives a backward traction force, and the upper frame 21 experiences a reactive force from the upper dental arch's attempt to return to its original position. Consequently, the force-bearing surface of the lower frame 31 is oriented towards the tongue side, while the force-bearing surface of the upper frame 21 is positioned towards the lip side. Additionally, since the displacement of the upper frame 21 relative to the original position of the teeth is smaller, the reactive force on the upper frame 21 is less than that on the lower frame 31. Therefore, the larger force-bearing surface of the upper frame 21 is the first outer wall 212, and the larger force-bearing surface of the lower frame 31 is the fourth outer wall 313.

In this embodiment, the first material 4, being of lower hardness, is less capable of providing sufficient support. The parts of the outer walls 212, 213, 312, 313 that contact the oral cavity are primarily made from the first material 4, and at least part of the outer walls 212, 213, 312, 313 include the second material 5. This means at least part of the first outer wall 212 and the second outer wall 213 include both the first material 4 and the second material 5. Similarly, at least part of the third outer wall 312 and the fourth outer wall 313 include both the first material 4 and the second material 5. The thickness of at least part of the outer walls 212, 213, 312, 313 is between 0.3 mm and 6 mm, as indicated in FIG. 11 as w3, with a preferred thickness of between 1.2 mm to 2 mm. The thickness of the second material 5 of the first outer wall 212, the second outer wall 213, the third outer wall 312, and the fourth outer wall 313 is 1 mm, as shown in FIG. 11 as w4. This design ensures the required support without being overly thick, and maintains the comfort afforded by the first material 4, thereby avoiding an increase in the weight of the mandibular advancement device 1 and reducing the burden on the oral cavity.

Given the different characteristics of the materials, the upper and lower tray assemblies 2 and 3 require different levels of support. In other implementations, the composition of the outer walls 212, 213, 312, 313 may vary, such as the upper frame 21 possibly lacking the first outer wall 212 and/or the second outer wall 213, or the lower frame 31 may not include the third outer wall 312 and/or the fourth outer wall 313.

### Embodiment 2

In this embodiment, the mandibular advancement device 1 includes an upper tray assembly 2 and a lower tray assembly 3. The upper tray assembly 2, configured to be adjacent to a portion of the user's upper dental arch, includes an upper frame 21 and an upper moldable component 22. The lower tray assembly 3, configured to be adjacent to a portion of the user's lower dental arch, includes a lower frame 31 and a lower moldable component 32. In this embodiment, the frames 21 and 31 include an upper frame 21 and a lower frame 31, and the moldable components 22 and 32 include an upper moldable component 22 and a lower moldable component 32.

Specifically, as shown in FIG. 2, the upper moldable component 22 and the lower moldable component 32 are configured to at least partially conform to the teeth and/or gums. The upper frame 21 and the lower frame 31 are each configured to engage and support the upper moldable component 22 and the lower moldable component 32, respectively. The upper frame 21 and the lower frame 31 include at least two materials of different hardness, and the hardness of the first material 4 is lower than that of the second material 5.

The upper frame 21 and the lower frame 31 include bottom walls 211, 311, and outer walls 212, 213, 312, 313. As depicted in FIGS. 4 and 6, the upper frame 21 includes a bottom wall 211, a first outer wall 212, and a second outer wall 213. The lower frame 31 includes a bottom wall 311, a third outer wall 312, and a fourth outer wall 313. The first outer wall 212 and the third outer wall 312 are configured to be oriented towards the lip side, while the second outer wall 213 and the fourth outer wall 313 are configured to be oriented towards the tongue side.

During the use of the mandibular advancement device 1, the lower tray assembly 3 advances relative to the upper tray assembly 2 to facilitate the forward movement of the mandible. During this process, the lower dental arch is subjected to a forward traction force, while the lower frame 31 experiences a reactive force from the lower dental arch's attempt to return to its original position. Conversely, the upper dental arch receives a backward traction force, and the upper frame 21 experiences a reactive force from the upper dental arch's attempt to return to its original position. Consequently, the force-bearing surface of the lower frame 31 is oriented towards the tongue side, while the force-bearing surface of the upper frame 21 is positioned towards the lip side. Additionally, since the displacement of the upper frame 21 relative to the original position of the teeth is smaller, the reactive force on the upper frame 21 is less than that on the lower frame 31. Therefore, the larger force-bearing surface of the upper frame 21 is the first outer wall 212, and the larger force-bearing surface of the lower frame 31 is the fourth outer wall 313.

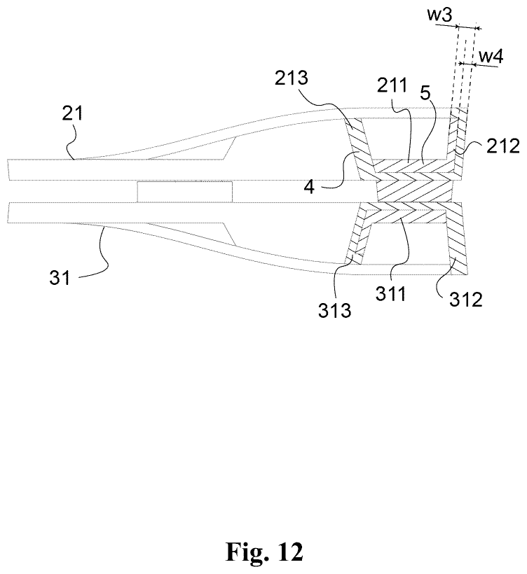

In this embodiment, the first material 4, while having some hardness to provide partial support, requires the second material 5 on the larger force-bearing surfaces for additional support. As shown in FIG. 12, the first outer wall 212 and the fourth outer wall 313 include both the second material 5 and the first material 4, whereas the second outer wall 213 and the third outer wall 312 only include the first material 4. On the larger force-bearing surfaces, the outer walls 212 and 313 include both the second material 5 and the first material 4 to provide adequate support; on the smaller force-bearing surfaces, the first material 4 alone is sufficient to support the outer walls 213 and 312. The thickness of at least part of the outer walls 212, 213, 312, 313 is between 0.3 mm to 6 mm, as indicated in FIG. 12 as w3, with a preferred thickness between 1.2 mm to 2 mm. The thickness of the second material 5 of the first outer wall 212 and the fourth outer wall 313 is 1 mm, as shown in FIG. 12 as w4. This design ensures sufficient support without being overly bulky, maintaining the comfort afforded by the first material 4 to the oral cavity, thereby avoiding increasing the weight of the mandibular advancement device 1 and reducing the burden on the oral cavity.

### Embodiment 3

In this embodiment, the mandibular advancement device 1 includes an upper tray assembly 2 and a lower tray assembly 3. The upper tray assembly 2, configured to be adjacent to a portion of the user's upper dental arch, includes an upper frame 21 and an upper moldable component 22. The lower tray assembly 3, configured to be adjacent to a portion of the user's lower dental arch, includes a lower frame 31 and a lower moldable component 32. In this embodiment, the frames 21 and 31 include an upper frame 21 and a lower frame 31, and the moldable components 22 and 32 include an upper moldable component 22 and a lower moldable component 32.

Specifically, as shown in FIG. 2, the upper moldable component 22 and the lower moldable component 32 are configured to at least partially conform to the teeth and/or gums. The upper frame 21 and the lower frame 31 are each configured to engage and support the upper moldable component 22 and the lower moldable component 32, respectively. The upper frame 21 and the lower frame 31 include at least two materials of different hardness, and the hardness of the first material 4 is lower than that of the second material 5.

The upper frame 21 and the lower frame 31 include bottom walls 211, 311, and outer walls 212, 213, 312, 313. As depicted in FIGS. 4 and 6, the upper frame 21 includes a bottom wall 211, a first outer wall 212, and a second outer wall 213. The lower frame 31 includes a bottom wall 311, a third outer wall 312, and a fourth outer wall 313. The first outer wall 212 and the third outer wall 312 are configured to be oriented towards the lip side, while the second outer wall 213 and the fourth outer wall 313 are configured to be oriented towards the tongue side.

During the use of the mandibular advancement device 1, the lower tray assembly 3 advances relative to the upper tray assembly 2 to facilitate the forward movement of the mandible. During this process, the lower dental arch is subjected to a forward traction force, while the lower frame 31 experiences a reactive force from the lower dental arch's attempt to return to its original position. Conversely, the upper dental arch receives a backward traction force, and the upper frame 21 experiences a reactive force from the upper dental arch's attempt to return to its original position. Consequently, the force-bearing surface of the lower frame 31 is oriented towards the tongue side, while the force-bearing surface of the upper frame 21 is positioned towards the lip side. Additionally, since the displacement of the upper frame 21 relative to the original position of the teeth is smaller, the reactive force on the upper frame 21 is less than that on the lower frame 31. Therefore, the larger force-bearing surface of the upper frame 21 is the first outer wall 212, and the larger force-bearing surface of the lower frame 31 is the fourth outer wall 313.

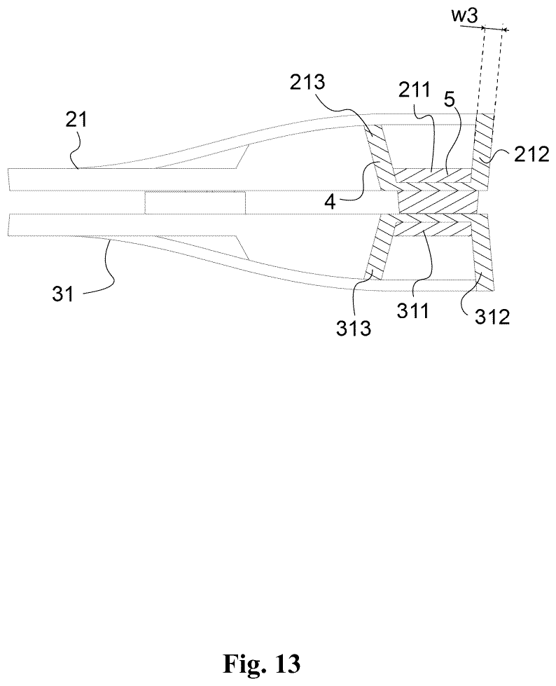

In this embodiment, the first material 4 possesses significant hardness and can provide sufficient support. As shown in FIG. 13, the first outer wall 212, the second outer wall 213, the third outer wall 312, and the fourth outer wall 313 include only the first material 4. The thickness of at least part of the outer walls 212, 213, 312, 313 is between 0.3 mm and 6 mm, as indicated in FIG. 13 as w3, with a preferred thickness of between 1.2 mm to 2 mm. This design ensures the necessary support without being overly bulky and maintains the comfort provided by the first material 4 to the oral cavity, thus avoiding increasing the weight of the mandibular advancement device 1 and reducing the burden on the oral cavity.

Furthermore, it is possible to combine the technical features described in the above embodiments as needed to obtain a mandibular advancement device 1 that includes all or some of these technical features.

The implementation of a mandibular advancement device provided by this disclosure at least includes the following benefits:
1. Existing mandibular advancement devices on the market typically use a frame made from a single material. Frames made solely from rigid materials can scrape against the inside of the mouth during wear, causing irritation or even injury to the soft tissues or the tongue. Additionally, if the entire mandibular advancement device is made from moldable material or other softer materials, it may not provide sufficient support during biting. Moreover, the lower tray assembly needs to move forward relative to the upper tray assembly, and they must be fixed together. Softer materials may deform, potentially changing the forward distance, which can lead to separation of the upper and lower tray assemblies and affect the treatment outcome. To address this issue, this disclosure adopts an innovative design: the frame includes at least two materials of different hardness. The outer wall of the frame is primarily made from the first material, which is softer and helps reduce scraping against the inside of the mouth during wear. The bottom wall of the frame is at least partially made from the second material, which is harder and provides the necessary support and stability during the biting process. Furthermore, the outer wall of the frame forms a certain angle with the horizontal plane to accommodate the natural inclination of the front teeth towards the lip side, fitting the shape of the teeth more closely. This design not only ensures support during the biting process but also securely connects the upper and lower tray assemblies, reducing the possibility of the device falling out, thereby ensuring good therapeutic effects during treatment.
2. When the user wears the mandibular advancement device, the lower tray assembly moves forward relative to the upper tray assembly to achieve the effect of mandibular advancement. However, this advancement can lead to crowding inside the mouth, especially when the forward distance is large, potentially causing contact with the deeper parts of the mouth and the frame, leading to a sensation of soreness and foreign body presence. Therefore, the outer walls of the frame are mostly made from the first material. Compared to traditional rigid materials, this first material is softer and more deformable, allowing it to better conform to the internal shape of the mouth, thus providing a more comfortable wearing experience. Additionally, the ends of the frame also use the softer first material to ensure comfort around the jaw joints and/or deeper parts of the mouth. This design helps enhance the comfort of wearing the mandibular advancement device, reduces discomfort for the user, improves the user experience, and thereby better enhances the therapeutic effects.
3. Mandibular advancement devices typically need to be worn for extended periods during sleep, and prolonged wearing and biting can cause discomfort or soreness in the user's mouth or teeth. Additionally, during the use of the mandibular advancement device, the lower tray assembly moves forward relative to the upper tray assembly to achieve the mandibular advancement effect. During this forward movement, the lower dental arch is subjected to forward traction forces, while the lower tray assembly experiences a reactive force from the lower dental arch's tendency to return to its original position; similarly, the upper dental arch is subjected to backward traction forces, while the upper tray assembly experiences a reactive force from the upper dental arch's tendency to return to its original position. Consequently, the larger force-bearing surface of the lower tray assembly is oriented towards the tongue side, and the larger force-bearing surface of the upper tray assembly is oriented towards the lip side. The present disclosure offers an improved frame design, and the outer wall of the frame is made from different materials depending on the direction of the force, with most parts that are likely to contact soft tissues of the mouth being made from the softer first material. Compared to the traditional design that uses a single rigid material, this design provides a more comfortable force-bearing surface, alleviating discomfort in the mouth and teeth, thereby enhancing the user's experience.
4. Existing mandibular advancement devices on the market still suffer from issues of dislodgement, with numerous user feedback indicating that the devices frequently separate from the mouth during sleep, leading to the device falling off. Testing and analysis have revealed that this dislodgement issue is primarily due to the requirement for the lower tray assembly to move forward relative to the upper tray assembly, and the possibility of the user moving or unconsciously opening their mouth during sleep, which can cause the mandibular advancement device to dislodge. Moreover, the outer walls of market-available frames are mostly made from rigid materials, which tend to slide easily upon contact with the mouth, further increasing the likelihood of dislodgement. To address this issue, the present disclosure provides an improved frame design with an outer wall that includes the softer first material. The first material has a higher coefficient of friction compared to traditional rigid materials, thus increasing friction with the mouth. By increasing the friction, the mandibular advancement device can be more securely fixed in the mouth, reducing the likelihood of dislodgement, and enhancing the stability and sustainability of the device during sleep.

The technical features of the above embodiments can be freely combined. For brevity, not all possible combinations of these features are described here. However, as long as the combinations do not introduce contradictions, they should be considered within the scope outlined by this disclosure.

The embodiments described above represent only a few of the possible implementations of the disclosure. Although the descriptions are specific and detailed, they should not be understood as limiting the scope of this disclosure. It should be noted that for one skilled in the art, various modifications and improvements can be made without departing from the concept of the disclosure, and these are also considered within the scope of protection of this disclosure. Therefore, the scope of protection for this disclosure should be determined by the appended claims.

It must be noted that as used herein and in the appended claims, the singular forms “a”, “an”, and “the” include their plural equivalents, unless the context clearly dictates otherwise.

1. A mandibular advancement device, configured to prevent or reduce snoring and/or obstructive sleep apnea during sleep, comprising:
an upper tray assembly consisting of two layers configured to be adjacent to a portion of an upper dental arch of a user, comprising an upper frame and an upper moldable component; and
a lower tray assembly consisting of two layers configured to be adjacent to a portion of a lower dental arch of the user, comprising a lower frame and a lower moldable component,
wherein the upper frame has at least one bottom wall, a first outer wall, and a second outer wall, and the lower frame has at least one bottom wall, a third outer wall, and a fourth outer wall,
the first outer wall and the third outer wall configured to be adjacent to a lip of a user, and the second outer wall and the fourth outer wall configured to be adjacent to a tongue of a user,
the upper frame and the lower frame are each configured to engage and support the upper moldable component and the lower moldable component respectively, wherein only a part of the upper moldable component is disposed in a space formed by the first outer wall, the second outer wall, and the at least one bottom wall of the upper frame, and only a part of the lower moldable component is disposed in a space formed by the third outer wall, the fourth outer wall, and the at least one bottom wall of the lower frame,
the upper moldable component and the lower moldable component are configured to at least partially conform to teeth and/or gums,
the upper tray assembly and the lower tray assembly include at least three materials of different hardness including a first material, a second material, and a third material,
a hardness of the first material is less than a hardness of the second material,
the at least one bottom wall of the upper frame has a first end and a second end, between which at least part of the at least one bottom wall forms an arc, and
the at least one bottom wall of the lower frame has a third end and a fourth end, between which at least part of the at least one bottom wall forms an arc.

2. The mandibular advancement device according to claim 1, wherein a shape of the upper tray assembly is arcuate, U-shaped, or arch-shaped.

3. The mandibular advancement device according to claim 1, wherein a shape of the lower tray assembly is arcuate, U-shaped, or arch-shaped.

4. The mandibular advancement device according to claim 1, wherein the first end, the second end, the third end, and the fourth end include the first material.

5. (canceled)

6. A mandibular advancement device, configured to prevent or reduce snoring and/or obstructive sleep apnea during sleep, comprising:
an upper tray assembly consisting of two layers configured to be adjacent to a portion of an upper dental arch of a user, comprising an upper frame and an upper moldable component; and
a lower tray assembly consisting of two layers configured to be adjacent to a portion of a lower dental arch of the user, comprising a lower frame and a lower moldable component,
wherein the upper frame and the lower frame are each configured to engage and support the upper moldable component and the lower moldable component respectively,
the upper frame has at least one bottom wall, a first outer wall, and a second outer wall, and the lower frame has at least one bottom wall, a third outer wall, and a fourth outer wall,
the first outer wall and the third outer wall configured to be adjacent to a lip of a user, and the second outer wall and the fourth outer wall configured to be adjacent to a tongue of a user,
only a part of the upper moldable component is disposed in a space formed by the first outer wall, the second outer wall, and the at least one bottom wall of the upper frame, and only a part of the lower moldable component is disposed in a space formed by the third outer wall, the fourth outer wall, and the at least one bottom wall of the lower frame,
the upper tray assembly and the lower tray assembly include at least three materials of different hardness including a first material, a second material, and a third material,
a hardness of the first material is less than a hardness of the second material, and the at least one outer wall of each frame at least partially includes the first material.

7. The mandibular advancement device according to claim 6, wherein the at least one outer wall of the upper and lower frames includes only the first material.

8. The mandibular advancement device according to claim 6, wherein, in a portion of the at least one outer wall of the upper and lower frames configured to be in contact with an oral cavity, an area of the first material is greater than an area of the second material.

9. The mandibular advancement device according to claim 6, wherein the first material includes at least one material selected from the group consisting of silicone rubber, polyvinyl alcohol, polyurethane, ethylene-vinyl acetate, thermoplastic olefin, thermoplastic elastomer, thermoplastic rubber, polylactic acid, and polylactic-co-glycolic acid.

10. The mandibular advancement device according to claim 6, wherein the second material includes at least one material selected from the group consisting of polycarbonate, polypropylene, acrylonitrile butadiene styrene, polyethylene, polymethyl methacrylate, polyurethane, stainless steel, titanium alloy, nickel-titanium alloy, and aluminum alloy.

11. (canceled)

12. A mandibular advancement device, configured to prevent or reduce snoring and/or obstructive sleep apnea during sleep, comprising:
an upper tray assembly consisting of two layers configured to be adjacent to a portion of an upper dental arch of a user, comprising an upper frame and an upper moldable component; and
a lower tray assembly consisting of two layers configured to be adjacent to a portion of a lower dental arch of the user, comprising a lower frame and a lower moldable component,
wherein the upper frame and the lower frame are each configured to engage and support the upper moldable component and the lower moldable component respectively,
the upper frame has at least one bottom wall, a first outer wall, and a second outer wall, and the lower frame has at least one bottom wall, a third outer wall, and a fourth outer wall,
the first outer wall and the third outer wall configured to be adjacent to a lip of a user, and the second outer wall and the fourth outer wall configured to be adjacent to a tongue of a user,
only a part of the upper moldable component is disposed in a space formed by the first outer wall, the second outer wall, and the at least one bottom wall of the upper frame, and only a part of the lower moldable component is disposed in a space formed by the third outer wall, the fourth outer wall, and the at least one bottom wall of the lower frame,
the upper tray assembly and the lower tray assembly include at least three materials of different hardness including a first material, a second material, and a third material,
a hardness of the first material is less than a hardness of the second material, and
the at least one bottom wall of each frame at least partially includes the second material.

13. The mandibular advancement device according to claim 12, wherein the at least one bottom wall of the upper and lower frames includes only the second material.

14. The mandibular advancement device according to claim 12, wherein a thickness of the second material of the at least one bottom wall of the upper and lower frames is at least 0.3 mm.

15. The mandibular advancement device according to claim 12, wherein a projected area of the at least one bottom wall of the upper and lower frames on a horizontal plane is at least 9 cm².

16. The mandibular advancement device according to claim 12, wherein the first material and the second material are joined by methods including at least one selected from the group consisting of crimping, over-molding, snap-fitting, bonding, thermal fusion, screws, and hooks.

17. A mandibular advancement device, configured to prevent or reduce snoring and/or obstructive sleep apnea during sleep, comprising:
an upper tray assembly consisting of two layers configured to be adjacent to a portion of an upper dental arch of a user, comprising an upper frame and an upper moldable component; and
a lower tray assembly consisting of two layers configured to be adjacent to a portion of a lower dental arch of the user, comprising a lower frame and a lower moldable component,
wherein the upper frame and the lower frame are each configured to engage and support the upper moldable component and the lower moldable component respectively,
the upper tray assembly and the lower tray assembly include at least three materials of different hardness including a first material, a second material, and a third material,
a hardness of the first material is less than a hardness of the second material,
the upper frame has at least one bottom wall, a first outer wall, and a second outer wall, and the lower frame has at least one bottom wall, a third outer wall, and a fourth outer wall,
the first outer wall and the third outer wall configured to be adjacent to a lip of a user, and the second outer wall and the fourth outer wall configured to be adjacent to a tongue of a user,
the at least one bottom wall of the upper frame has a first end and a second end, between which at least part of the at least one bottom wall of the upper frame forms an arc,
the at least one bottom wall of the lower frame has a third end and a fourth end, between which at least part of the at least one bottom wall of the lower frame forms an arc,
the upper frame and the lower frame have one or more of the following characteristics:
a thickness of at least part of the first outer wall, the second outer wall, the third outer wall, or the fourth outer wall each is between 0.3 mm to 6 mm; and
a thickness of at least part of the at least one bottom wall is between 0.3 mm to 6 mm.

18. The mandibular advancement device according to claim 17,
wherein when a middle of the upper frame is defined as a mid-point between the first end and the second end of the at least one bottom wall of the upper frame along a curve of the arc, and a middle of the lower frame is defined as a mid-point between the third end and the fourth end of the at least one bottom wall of the lower frame along a curve of the arc, a height of at least one of the outer walls at the middle of the upper frame or the lower frame is greater than a height of said at least one of the outer walls at ends of the said at least one of the outer walls.

19. The mandibular advancement device according to claim 17,
wherein when a middle of the upper frame is defined as a mid-point between the first end and the second end of the at least one bottom wall of the upper frame along a curve of the arc, and a middle of the lower frame is defined as a mid-point between the third end and the fourth end of the at least one bottom wall of the lower frame along a curve of the arc,
a distance between the first outer wall and the second outer wall at of the middle of the upper frame is less than the distance between the first outer wall and the second outer wall at the first end or the second end of the upper frame, or
a distance between the third outer wall and the fourth outer wall at the middle of the lower frame is less than the distance between the third outer wall and the fourth outer wall at the third end or the fourth end of the lower frame.

20. (canceled)

21. The mandibular advancement device according to claim 17, wherein the second outer wall includes only the first material, and the first outer wall includes the second material.

22. The mandibular advancement device according to claim 17, wherein the third outer wall includes only the first material, and the fourth outer wall includes the second material.

23. The mandibular advancement device according to claim 17, wherein a volume ratio of the first material to the second material is between 0.05 to 20.
